(19) United States
(12) Patent Application Publication
(10) Pub. No.: US 2010/0191474 A1
(43) Pub. Date: Jul. 29, 2010

(54) ELECTRONIC NOSE DEVICE WITH SENSORS COMPOSED OF NANOWIRES OF COLUMNAR DISCOTIC LIQUID CRYSTALS WITH LOW SENSITITIVE TO HUMIDITY

(75) Inventor: Hossam Haick, Haifa (IL)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(73) Assignee: TECHNION RESEARCH AND DEVELOPMENT FOUNDATION LTD., Haifa (IL)

(21) Appl. No.: 12/682,658

(22) PCT Filed: Oct. 23, 2008
(86) PCT No.: PCT/IL08/01404

§ 371 Date: Apr. 12, 2010

Related U.S. Application Data
(60) Provisional application No. 60/981,828, filed on Oct. 23, 2007.

(51) Int. Cl. G01N 33/48; G06F 19/00; G01N 27/00; G06N 3/02; G06N 3/12; G06N 7/02; G06N 5/04; G06F 15/18

(52) U.S. Cl. 702/19; 702/32; 324/71.1; 706/20; 706/13; 706/2; 706/52; 706/14

(57) ABSTRACT

Electronic nose device having a plurality of sensors composed of nanowires of columnar discotic liquid crystals, the device having low sensitivity to humidity. The device is designed to determine the composition and concentration of volatile organic compounds in a sample with very high sensitivity. Methods for use of the device in applications such as diagnosis of disease and food quality control are disclosed.

### FIELD OF THE INVENTION

The present invention relates to an electronic device for the detection of minute concentrations of volatile organic compounds with low sensitivity to humidity. In particular, the device of the invention comprises sensors of discotic liquid crystals in a nanowire configuration designed for the diagnosis of various disease biomarkers.

### BACKGROUND OF THE INVENTION

Electronic nose devices perform odor detection through the use of an array of cross-reactive sensors in conjunction with pattern recognition algorithms. In contrast to the “lock-and-key” model, each sensor in the electronic nose device is widely responsive to a variety of odorants. In this architecture, each analyte produces a distinct fingerprint from an array of broadly cross-reactive sensors. This configuration allows to considerably widen the variety of compounds to which a given matrix is sensitive, to increase the degree of component identification and, in specific cases, to perform an analysis of individual components in complex multi-component (bio) chemical media. Pattern recognition algorithms can then be applied to the entire set of signals, obtained simultaneously from all the sensors in the array, in order to glean information on the identity, properties and concentration of the compounds to be detected.

Micro-organisms produce patterns of volatile organic compounds (VOCs) that are affected by the type and age of culture. These can be used as biomarkers for detecting food spoilage as well as biomarkers of various diseases. Some examples for electronic nose devices for the detection of VOCs that are applicable for the food industry are disclosed in U.S. Pat. Nos., 4,399,687, 6,170,318, 6,234,006, 6,428,748, 6,537,802, 6,658,915, 6,767,732, 6,837,095 and US Patent Application Nos. 2006/0078658 and 2006/0191319. VOCs as biomarkers for a variety of diseases can be found, inter alia, in serum, urea, and breath. More than a thousand different VOCs have been detected in normal human breath (Phillips, Anal. Biochem., 247, 272, 1997). When diseases such as lung cancer, liver disease, inflammatory bowel disease, and rheumatoid arthritis appear, an increase in the level of oxidative stress markers can be observed (Phillips et al., J. Chromatogr. B. Biomed. Sci. ASSl, 729, 75, 1999; Phillips et al., J. Lab Clin. Med., 136, 243, 2000). The characteristic VOCs of a specific disease display different patterns at different stages of the disease.

Various odor detecting devices and systems employing sensor arrays that can be used in medical applications are known in the art. Some examples are disclosed in U.S. Pat. Nos. 6,173,602, 6,319,724, 6,411,905, 6,467,333, 6,540,691, 6,609,068, 6,620,109 6,703,241, 6,839,636, 6,841,391 7,052,854, 7,153,272, 7,241,989; and in US Patent Application Nos. 2001/0041366, 2002/0127623, 2005/0037374, 2005/0065446, 2006/0151687, 2006/0160134, 2006/0277974 and 2007/0062255.

The use of Gas-Chromatography linked with Mass-Spectrometry (GC-MS), Quartz Crystal Microbalance (QCM) as well as other pertinent instruments for analysis of these volatile disease biomarkers is impeded by several factors. These factors include the use of expensive equipment, the degree of expertise required to operate such instruments, the length of time required for data acquisition, and other technical problems in sampling, analysis of data, etc. Most importantly, the majority of the devices are limited to detection of analytes in the range of 1-100 parts per million (ppm) whereas many disease biomarkers are found at the parts per billion (ppb) level of concentrations. For targeting such low levels of VOCs, either pre-concentrating the sample prior to performing a measurement or acquiring data for longer time intervals are required.

Another problem encountered in the diagnosis of diseases through the analysis of VOCs in breath using existing devices, is the sensitivity of these devices to the relatively high humidity (˜80%) of breath samples.

Liquid crystals form an intermediate state between liquids and crystals. They thus share both some of the positional and orientational order of solid crystals, as well as some of the fluid behavior of liquids. The characterization of liquid crystals is performed according to the manner in which they are ordered. Liquid crystals are usually characterized as being ordered along a single dimension, where the extent of ordering rarely exceeds the macroscopic scale. Other dimensions are essentially disordered.

Known to date are a variety of liquid crystalline phases, also termed mesophases. In general, they can be divided into two categories: thermotropic and lyotropic exhibiting liquid crystalline properties at a certain temperature range or at a certain concentration range, respectively. The thermotropic liquid crystals can be further divided into sub-categories according to the shape adopted by the constituent molecules. Discotic liquid crystals are comprised of compounds having disc-like molecular shape (Chandrasekhar et al., Pramana, 9, 471, 1977). The disc-shaped molecules can be self-assembled to form mesophase structures with either a fluid nematic or a more viscous columnar arrangement. The nematic phase is characterized by orientational order, namely, the disc-shaped macrocycles have orientational correlation solely and no positional correlation (Höger, Chem. Eur. J., 10, 1320, 2004). In the columnar phase the disc-shaped molecules are stacked one on top of another to form a 2D lattice type of structure. The columns can adopt a hexagonal, rectangular, oblique, plastic, helical or lamellar packing (reviewed in Kumar, Chem. Soc. Rev., 35, 83, 2006).

Columnar discotic liquid crystals generally consist of an electron-rich aromatic core (a disc-like mesogen) which forms the columns, surrounded by insulating aliphatic side chains. The columnar arrangement of the discotic molecules allows energy migration due to the overlap of π-electron orbitals between neighboring aromatic cores. Thus, the columns act like molecular wires with relatively high charge mobility along the columns (Ohta et al., Mol. Cryst. Liq. Cryst., 397, 25, 2003). In contrast, carrier mobility is negligible perpendicular to the columns due to the intervening hydrocarbon chain regions which hinder the π-πinteractions between neighboring columns. Measurements of the charge carrier mobility in fabricated devices of homeotropically aligned discotic liquid crystals verified the highly anisotropic nature of semiconducting DLC films, wherein the mobility along the columns of discotic molecules was found to be at least two orders of magnitude higher than the mobility perpendicular to the columns (Deibel et al., Org. Electr., 7, 495, 2006; arXiv:cond-mat/0607392v1).

Discotic liquid crystals can be fabricated to a nanowire configuration wherein the molecules self-align into columnar molecular stacks surrounded by entangled aliphatic chains. In this configuration, the columns are semiconducting wires. The nanowires can be aligned using optical microscopy and conventional photolithographic processing (reviewed by Dorneanu, Softpedia™, Apr. 30, 2007).

Many discotic liquid crystals have been found applicable mostly as electro-optical display devices. Other uses include one-dimensional conductors, light emitting diodes (LED), field-effect transistors etc. Boden et al. (Handbook of Liq. Cryst. 2B, Chap. IX, 1998) showed that discotic liquid crystals can be used as fluid and gas sensors. U.S. Pat. No. 6,423,272 discloses the use of DLC in a 2D film configuration for the detection of fluids and gases and the applicability of such films for process control of chemical reactions and for environmental control. US Patent Application Nos. 2006/0210436 and 2007/0036681 demonstrate the use of nematic liquid crystal in a thin film configuration for vapor sensing. It is to be noted that the above-mentioned patents and patent applications use organic molecules that incorporate elements other than hydrocarbons to construct the liquid crystalline mesophases. These elements are typically positioned at the surface of the films where the interaction with guest molecules occurs. The structure as well as composition of these liquid crystalline mesophases, therefore, tends to be highly sensitive to humid environments. Furthermore, the hitherto known devices use discotic liquid crystals in a thin film homeotropical configuration which is more sensitive to diffusion effects.

Thus, there is yet an unmet need for highly sensitive reliable device to analyze mixtures of VOCs, with low sensitivity to humidity. Furthermore, there is an unmet need for inexpensive, efficient, convenient, and portable devices to analyze mixtures of VOCs in real time without pre-concentrating or dehumidifying the sample.

### SUMMARY OF THE INVENTION

The present invention provides an electronic device comprising at least one, preferably a plurality of nanowires of columnar discotic liquid crystals for the detection of volatile organic compounds (VOCs). In particular, the electronic device of the present invention has low susceptibility towards humidity, and is thus more sensitive than known systems serving a similar purpose. The present invention further provides a system comprising an electronic device comprising an array of chemically sensitive sensors of columnar discotic liquid crystal nanowires in conjunction with pattern recognition analyzer, wherein the pattern recognition analyzer uses methods such as artificial neural networks and principal component analysis to detect as well as quantify specific volatile organic compounds, without a need for sample pre-processing steps.

The invention is based in part on the unexpected finding that wires of columnar discotic liquid crystals can be used as sensors with improved sensing capabilities and essentially no sensitivity to humidity. The low sensitivity to humidity is achieved through the use of columnar discotic liquid crystals having a nanowire configuration wherein the surface of the nanowires is composed of hydrocarbons. In this unique configuration, elements that might be sensitive to humidity (e.g. oxygen, nitrogen), if present, are all confined to the core of the self-assembled nanowires. The devices disclosed herein enable the detection of minute quantities of volatile organic compounds as biomarkers for diagnostic and prognostic purposes without the need for pre-concentrating or dehumidifying the sample.

According to one aspect, the present invention provides an electronic device comprising at least one chemically sensitive sensor for the detection of volatile organic compounds (VOCs), wherein the chemically sensitive sensor comprises at least one nanowire of columnar discotic liquid crystals, and wherein the electronic device is essentially not sensitive to humidity.

According to another aspect, the present invention provides a system for detecting VOCs comprising an electronic device which comprises an array of sensors of columnar discotic liquid crystal nanowires, and a pattern recognition analyzer, wherein the pattern recognition analyzer receives sensor signal outputs and compares them to stored data.

In one embodiment, the electronic devices of the present invention can be used in a configuration selected from the group consisting of chemiresistor, chemicapacitor, and Field Effect Transistor (FET).

In another embodiment, the electronic devices of the present invention detect VOCs with sensitivity of less than one part per million (ppm). In a currently preferred embodiment, the electronic devices disclosed herein detect volatile organic compounds with sensitivity of 100 parts per billion (ppb), or less.

In some embodiments, the electronic devices of the present invention comprise the columnar discotic liquid crystal nanowires in an edge-on configuration (planar orientation). In various embodiments, the columnar discotic liquid crystal nanowires of the present invention are processed in a top-down approach. In alternative embodiments, the columnar discotic liquid crystal nanowires of the present invention are manufactured in a bottom-up approach.

According to the principles of the present invention, the columnar discotic liquid crystals may be self assembled to form meso- and macrowires having diameters in the range of 1-20 μm and lengths in the range of 0.01-800 μm. In currently preferred embodiments, the columnar discotic liquid crystal nanowires have diameters in the range of 5-500 nm and lengths in the range of 0.01-500 μm.

According to certain embodiments, the columnar discotic liquid crystal nanowires of the present invention are largely insensitive to humidity. This low sensitivity to humidity is achieved by the use of molecules composed mainly of hydrocarbons. In certain embodiments wherein any heteroatom other than hydrogen and carbon is present, the heteroatom that might be sensitive to humidity is confined to the core of the self-assembled nanowire.

According to some embodiments, the columnar discotic liquid crystal nanowires are assembled from molecules selected from the group consisting of: hexa-n-alkoxytriphenylenes [C₁₈H₆(—O—CₙH₂ₙ₊₁)₆], hexa-n-alkylthiotriphenylenes [C₁₈H₆(—S—CₙH₂ₙ₊₁)₆], and hexa-n-alkoxyaryltriphenylenes [C₁₈H₆(C₆H₃(—O—CₙH₂ₙ₊₁)₂]₆], wherein n=5, 6, 8, 11, or 13.

According to various embodiments, the columnar discotic liquid crystal nanowires are assembled from hexa-alkyl-substituted derivatives of hexabenzo[bc,ef,hi,kl,no,qr]coronene [C₄₂H₁₂—(CₙH₂₊₁)₆], wherein n=5, 6, 8, or 11.

According to other embodiments, the columnar discotic liquid crystal nanowires are assembled from hexa-alkylaryl-substituted derivatives of hexabenzo-[bc,ef,hi,kl,no,qr]coronene [C₄₂H₁₂(C₆H₄(—CₙH₂ₙ₊₁))₆], wherein n=5, 6, or 12.

According to yet other embodiments, the columnar discotic liquid crystal nanowires are assembled from molecules selected from the group consisting of: 2,3,6,7,10,11-hexakis[3,4-bis(alkoxy)-phenyl]triphenylenes [C₁₈H₆(C₆H₃(—O—CₙH₂ₙ₊₁)₂)₆], 6,6′,6″,7,7′,7″,10,10′,10″,11,-11′,11″-dodecaalkoxy-2,3′:3,2″:2′,3″tris(triphenylenylenes) [C₅₄H₁₈(—OCₙH₂ₙ₊₁)₁₂], wherein n=6, 8, 10, and 12.

According to currently preferred embodiments, the columnar discotic liquid crystal nanowires are assembled from hexa-peri-hexabenzocoronene (HBC) molecules terminated with any one of 2-ethyl-hexyl (HBC-C_{6,2}), 2-hexyl decane (HBC-C_{10,6}), 2-decyl tetradecane (HBC-C_{14,10}), and dodecane (HBC-C₁₂).

According to another aspect, the present invention further provides a system comprising an electronic device for detecting VOCs, wherein the electronic device comprises an array of chemically sensitive sensors comprising nanowires of columnar discotic liquid crystals in conjunction with pattern recognition analyzer, wherein the pattern recognition analyzer receives sensor output signals and compares them to stored data. The pattern recognition analyzer may utilize various algorithms including algorithms based on artificial neural networks, multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART) and statistical methods such as principal component analysis (PCA), partial least squares (PLS), multiple linear regression (MLR), principal component regression (PCR), discriminant function analysis (DFA) including linear discriminant analysis (LDA), and cluster analysis including nearest neighbor.

According to another aspect, the present invention provides a method of determining at least one of the composition and concentration of selected VOCs in a sample using the system of the present invention, comprising the steps of: (a) providing a system comprising an electronic device for detecting VOCs comprising an array of chemically sensitive sensors of columnar discotic liquid crystal nanowires, and a pattern recognition analyzer, wherein the pattern recognition analyzer receives sensor output signals and compares them to stored data, (b) exposing the sensor array of the electronic device to the sample, and (c) using pattern recognition algorithms to detect the presence of the VOCs and preferably measure their concentration in the sample.

According to particular embodiments, the method of determining selected VOCs in a sample using the system of the present invention comprises detecting the VOCs through spectroscopic ellipsometry.

According to yet another aspect, the present invention provides a method for diagnosing a disease in a subject by determining at least one of the composition and concentration of disease biomarkers in a sample, using the system of the present invention, comprising the steps of: (a) collecting a sample comprising VOCs selected from exhaled breath and the headspace of a container in which at least one bodily fluid or secretion of the subject has been placed, (b) providing a system comprising an electronic device for detecting VOCs comprising an array of chemically sensitive sensors of columnar discotic liquid crystal nanowires, and a pattern recognition analyzer, wherein the pattern recognition analyzer receives sensor output signals and compares them to stored data, (c) exposing the sensor array of the electronic device to the sample, and (d) using pattern recognition algorithms to determine the composition and preferably measure the concentration of selected VOCs indicative of a disease in the sample.

In certain aspects, the present invention relates to the use of an electronic device comprising an array of chemically sensitive sensors wherein the chemically sensitive sensors comprise columnar discotic liquid crystal nanowires, and a pattern recognition analyzer, wherein the pattern recognition analyzer receives sensor output signals and compares them to stored data, for the preparation of an apparatus for detecting VOCs. In a currently preferred embodiment, the use disclosed herein is designated towards detecting VOCs that are indicative of a disease in a subject.

The present invention further provides a system for diagnosing a disease in a subject comprising exposing an electronic device comprising an array of chemically sensitive sensors wherein the chemically sensitive sensors comprise columnar discotic liquid crystal nanowires to the breath of a subject, or to the headspace of a container in which a bodily fluid of the subject has been deposited, and using pattern recognition algorithms to receive sensor output signals and compare them to stored data.

Bodily fluids or secretions that can be tested according to the principles of the present invention include, but are not limited to, serum, urine, feces, sweat, vaginal discharge, saliva and sperm.

Within the scope of the present invention is the diagnosis of a disease or disorder selected from the group consisting of: acute asthma, hepatic encephalopathy, rheumatoid arthritis, schizophrenia, ketosis, cardiopulmonary disease, uremia, diabetes mellitus, larynx cancer, dysgeusia/dysosmia, cystinuria, cirrhosis, histidinemia, tyrosinemia, halitosis and phenylketonuria. According to a currently preferred embodiment, the present invention provides a method to diagnose cancer.

According to some aspects, the present invention provides a method to detect spoilage in food products via the determination of at least one of the composition and concentration of VOC in a food sample, using the system of the present invention, comprising the steps of: (a) collecting a sample of VOCs from the headspace of a container in which a food product has been placed, (b) providing a system comprising an electronic device for detecting VOCs comprising an array of chemically sensitive sensors of columnar discotic liquid crystal nanowires, and a pattern recognition analyzer, wherein the pattern recognition analyzer receives sensor output signals and compares them to stored data, (c) exposing the sensor array of the electronic device to the sample, and (d) using pattern recognition algorithms to determine the composition and preferably measure the concentration of selected VOCs indicative of spoilage.

Encompassed within the scope of the present invention is the use of an electronic device comprising an array of chemically sensitive sensors wherein the chemically sensitive sensors comprise columnar discotic liquid crystal nanowires, and a pattern recognition analyzer, wherein the pattern recognition analyzer receives sensor output signals and compares them to stored data, for the preparation of an apparatus for detecting spoilage in food products via the determination of at least one of the composition and concentration of VOCs in a food sample.

In some embodiments, the present invention provides a system for detecting spoilage in food products via the determination of at least one of the composition and concentration of VOCs in a food sample, comprising exposing an electronic device comprising an array of chemically sensitive sensors wherein the chemically sensitive sensors comprise columnar discotic liquid crystal nanowires to the sample, and using pattern recognition algorithms to receive sensor output signals and compare them to stored data.

According to other aspects, the present invention provides a method for detecting pollutants in water or air for environmental monitoring comprising the steps of: (a) collecting a sample of water vapor or air to a container, (b) providing a system comprising an electronic device comprising an array of chemically sensitive sensors of columnar discotic liquid crystal nanowires, and a pattern recognition analyzer, wherein the pattern recognition analyzer receives sensor output signals and compares them to stored data, (c) exposing the sensor array of the electronic device to the sample, and (d) using pattern recognition algorithms to determine the composition and preferably measure the concentration of pollutants molecules in water or air.

In specific embodiments, the present invention provides the use of an electronic device comprising an array of chemically sensitive sensors wherein the chemically sensitive sensors comprise columnar discotic liquid crystal nanowires, and a pattern recognition analyzer, wherein the pattern recognition analyzer receives sensor output signals and compares them to stored data, for the preparation of an apparatus for detecting pollutants in water or air.

In other embodiments, the present invention provides a system for detecting pollutants in water or air, comprising exposing an electronic device comprising an array of chemically sensitive sensors wherein the chemically sensitive sensors comprise columnar discotic liquid crystal nanowires to a sample of water or air, and using pattern recognition algorithms to receive sensor output signals and compare them to stored data.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 is a schematic diagram illustrating the differentiation between odorants using an array of broadly-cross reactive sensors, in which each individual sensor responds to a variety of odorants, in conjugation with pattern recognition algorithms to allow classification. ‘A’—raw measurements, ‘B’—normalized measurements, ‘C’—feature vector, ‘D’—odor class (confidence level), ‘E’—post processed odor class, ‘F’—decision making, ‘G’—classification, ‘H’—dimensionality reduction, and ‘I’—signal preprocessing.

FIGS. 2A-2F are Scanning Electron Microscopy (SEM) micrographs of exemplary DLC nanowires assembled at different experimental conditions.

FIG. 3 is a schematic representation of the sensing device, constructed from at least one, preferably a plurality of nanowires of columnar discotic liquid crystals, laid upon a dielectric layer. ‘A’ represents analyte molecules, and ‘S’ represents sensing molecules.

FIG. 4 is a Scanning Electron Microscopy (SEM) micrograph of a 1.5 μm in diameter DLC wire that is made of edge-to-edge self-assembled 50 nm (in diameter) DLC nanowires.

FIG. 5 is an Atomic Force Microscope (AFM) micrograph of a DLC nanowire. The nanowire is laid down in an edge-on configuration (planar orientation). X range and Y range correspond each to 2 μm.

FIG. 6 is a Scanning Electron Microscope (SEM) micrograph of a 50 nm in diameter DLC nanowire that is contacted by means of Focused Ion Beam (FIB) to form Field Effect Transistor (FET).

FIG. 7 is a Voltage (V)-current (I) correlation of a field effect transistor based on a DLC nanowire, showing no sensitivity to varying humidity environments, at different exposure times.

FIG. 8 is a Scanning Electron Microscope (SEM) micrograph of the HBC-C₁₋₂ structures formed on a Si/SiO₂ surfaces.

FIG. 9 is a graph representing changes in “thickness” of HBC-C₁₂ layers during exposure to water, octane and decane analytes.

### DETAILED DESCRIPTION OF THE INVENTION

The present invention provides an electronic device comprising at least one sensor of discotic liquid crystal in a nanowire configuration for the detection of specific VOCs. The invention further provides a system comprising an array of sensors of discotic liquid crystal nanowires and pattern recognition analyzer which utilizes algorithms such as principal component analysis and neural networks. Further disclosed are methods for detecting and classifying certain biomarkers for diagnostic and prognostic purposes.

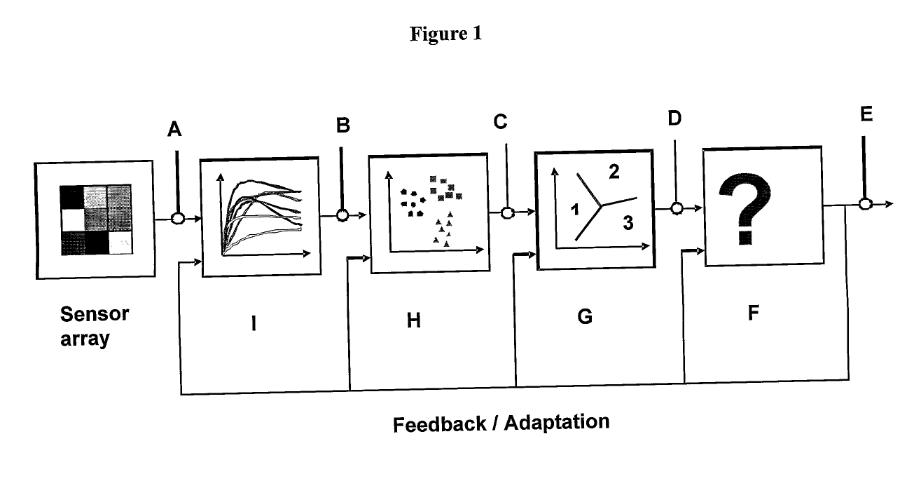

The electronic nose device presented herein provides odor detection through the use of an array of cross-reactive sensors in conjunction with pattern recognition algorithms. According to the principles of the present invention, sensing is obtained through adsorption of vapors to provide changes in electrical signal which are then conveyed to a pattern recognition analyzer to generate identification of desired VOCs. FIG. 1 schematically illustrates the differentiation between odorants using the electronic nose devices. Particularly, the array of sensors is exposed to a variety of VOCS to provide an electronic response vs. time (2^{nd} box on the left). The dimensionality is then reduced wherein the data is represented by a new basis set (f₂ vs. f₁; 3^{rd} box on the left). This representation allows to classify the different odors (1, 2 & 3; 4^{th} box on the left). The procedure can be iteratively performed until satisfactory odor classification is achieved.

Discotic liquid crystal (DLC) films, such as those disclosed e.g. in U.S. Pat. No. 6,423,272, exhibit different properties than the DLC nanowires of the present invention. The formation of DLC films is usually much less uniform having been assembled on the surface. In contrast, nanowires are assembled in a more controlled environment and are thus uniform in size exhibiting variability of less than 10%. It is hence possible to control the dimensions of the DLC nanowires by varying the concentration of DLC molecules in solution as well as the solvent used in the self-assembly process. Another manner of controlling the dimensions of the DLC nanowires is by varying the method used to coat the solid-state substrate (e.g., spin coating, spray coating, Langmuir-Blodgett, zone-casting, drop casting, etc).

In sensors comprising 2D films of DLCs, the electronic properties of the sensors are impaired by diffusion problems, thus lowering their sensitivity. Without being bound by any theory or mechanism of action, self-organized molecular dynamics results in the accumulation of charged and uncharged impurities near the surface of liquid crystals. This feature as well as the large surface to bulk ratio of nanowires in comparison to 2D films, renders nanowire configuration advantageous in preventing diffusion limitations of the vapor analytes into the bulk. Electronic devices composed of DLC nanowires thus provide fast on-off responses.

Furthermore, US Application No. 2004/0010028 discloses a three-dimensional molecular array on one of a conductor and a semiconductor surface, the array comprising at least one columnar stack comprising a plurality of substituted aromatic rings, wherein the aromatic rings of each columnar stack lie about parallel to the surface and the columnar stack comprises a plurality of hydrogen bonds between substituents of different rings. Application No. DE 19534494 discloses an electronic transistor device wherein the space between the electrodes is filled with columnar discotic crystals, which are aligned with their axes perpendicular to the electrodes. The molecules used in both applications incorporate elements other than hydrocarbons to construct the DLC structures.

In contrast to these references, the surface of the columnar DLC nanowires of the present invention consists essentially of hydrocarbons. This feature is of particular importance since the interaction with guest molecules occurs on the surface thus rendering these DLC nanowires essentially insensitive to humid environments. The nanowire configuration allows for elements that might be sensitive to humidity to be confined to the core of the self-assembled nanowires (see Bennett et al., J. Phys. Chem. C, ASAP Article, 10.1021/jp801476f; Saecker et al., Chem. Phys., 99, 7056, 1993). In this manner, the nanowire sensors possess very low sensitivity to humidity enabling the detection of biomarkers in human breath as well as in bodily fluids and secretions which may contain up to 80% relative humidity.

The term “low sensitivity to humidity” or “essentially no sensitivity to humidity” as used herein refers to minimum (or absence of) electrical responses from saturated level of water. Encompassed within the scope of the invention is the detection of VOCs and in particular apolar VOCs in a sample containing up to 100% relative humidity (RH). Minimum electronic responses to humidity correspond, according to the principles of the present invention, to responses of less than 25% of the responses to apolar VOCs in a sample saturated with water vapors. Preferably, the electronic responses to humidity would correspond to responses of less than 10% of the responses to apolar VOCs in a sample saturated with water vapors.

Without wishing to be bound by any theory or mechanism of action, it is believed that it is likely that adsorption of molecules on the surface produces changes in the switching field and, therefore, modifies the conductivity response. It stems from the hydrophobic nature of the discotic molecules of the present invention as well as from their unique nanowire configuration, that sensors built on these principles can detect VOCs with minor sensitivity to water vapor (humidity).

According to the principles of the present invention, the electronic devices comprise sensors of columnar DLC nanowires. The term “discotic liquid crystals” or “DLC” as used herein refers to a group of liquid crystals, which are comprised of compounds having disc-like molecular shape. The term “columnar discotic liquid crystals” as used herein refers to disc-shaped molecules, which are stacked one on top of the other to form a 2D lattice type of structure. The columns can adopt a hexagonal, rectangular, oblique, plastic, helical or lamellar packing. The term “nanowires” as used herein refers to DLC molecules arranged in a cylinder-like shape, having one of the dimensions elongated with respect to the other. It is to be understood that the term “nanowires” as used herein includes wires having dimensions in the nanometer as well as micrometer range. The term “nanowire” thus encompasses nanowires as well as microwires. Non-limiting examples are nanowires having a diameter perpendicular to the elongated dimension of about 5-500 nm and lengths in the range of about 0.01-500 μm, and nanowires having a diameter perpendicular to the elongated dimension of about 1-20 μm and lengths in the range of about 0.01-800 μm. This definition includes an ensemble of cylindrical nanowires having equivalent dimensions to the above mentioned, that are assembled edge-to-edge to form wires having essentially larger diameters. A cross-section along the elongated dimension can adopt either one of the following shapes: circular, trapezoidal, triangular, square, or rectangular.

In currently preferred embodiments, molecules disclosed hereinbelow are self-assembled to form the columnar DLC nanowires of the present invention. These molecules can be synthesized according to well-known procedures in the art. Molecules used for this purpose include, inter alia:

(I) hexa-n-alkoxytriphenylenes [C₁₈H₆(—O—CₙH₂ₙ₊₁)₆], hexa-n-alkylthiotriphenylenes [C₁₈H₆(—S—CₙH₂ₙ₊₁)₆], and hexa-n-alkoxyaryltriphenylenes [C₁₈H₆(C₆H₃(—O—CₙH₂ₙ₊₁)₆], wherein n=5, 6, 8, 11, or 13.
(II) hexa-alkyl-substituted derivatives of hexabenzo[bc,ef,hi,kl,no,qr]coronene [C₄₂H₁₂—(CₙH₂ₙ₊₁)₆], wherein n=5, 6, 8, or 11.
(III) hexa-alkylaryl-substituted derivatives of hexabenzo-[bc,ef,hi,kl,no,qr]coronene [C₄₂H₁₂(C₆H₄(—CₙH₂ₙ₊₁))₆], wherein n=5, 6, or 12.
(IV) 2,3,6,7,10,11-hexakis[3,4-bis(alkoxy)-phenyl]triphenylenes [C₁₈H₆(C₆H₃(—O—CₙH₂ₙ₊₁)₂)₆], 6,6′,6″,7,7′,7″, 10,10′,10″,11,11′,11″-dodecaalkoxy-2,3′:3,2″:2′,3″tris(triphenylenylenes) [C₅₄H₁₈(—OCₙH₂ₙ₊₁)₁₂], wherein n=6, 8, 10, and 12.
(V) hexa-peri-hexabenzocoronene (HBC) molecules having the following functional groups: HBC-C_{6,2}, HBC-C_{10,6}, HBC-C_{14,10}, and HBC-C₁₂.

Many more molecules that satisfy the definition of “discotic liquid crystals” may be used in the same context.

An “alkyl” group refers to a saturated aliphatic hydrocarbon, including straight-chain, branched-chain and cyclic alkyl groups. In one embodiment, the alkyl group has 1-12 carbons designated here as C₂-C₁₂-alkyl. In another embodiment, the alkyl group has 2-6 carbons designated here as C₂-C₆-alkyl. In another embodiment, the alkyl group has 2-4 carbons designated here as C₂-C₄-alkyl.

A “cycloalkyl” group refers to a non-aromatic mono- or multicyclic ring system. In one embodiment, the cycloalkyl group has 3-10 carbon atoms. In another embodiment, the cycloalkyl group has 5-10 carbon atoms. Exemplary monocyclic cycloalkyl groups include cyclopentyl, cyclohexyl, cycloheptyl and the like. An alkylcycloalkyl is an alkyl group as defined herein bonded to a cycloalkyl group as defined herein.

An “alkenyl” group refers to an aliphatic hydrocarbon group containing at least one carbon-carbon double bond including straight-chain, branched-chain and cyclic alkenyl groups. In one embodiment, the alkenyl group has 2-8 carbon atoms (a C₂₋₈ alkenyl). In another embodiment, the alkenyl group has 2-4 carbon atoms in the chain (a C₂₋₄ alkenyl). Exemplary alkenyl groups include ethenyl, propenyl, n-butenyl, i-butenyl, 3-methylbut-2-enyl, n-pentenyl, heptenyl, octenyl, cyclohexyl-butenyl and decenyl. An alkylalkenyl is an alkyl group as defined herein bonded to an alkenyl group as defined herein.

An “alkynyl” group refers to an aliphatic hydrocarbon group containing at least one carbon-carbon triple bond including straight-chain and branched-chain. In one embodiment, the alkynyl group has 2-8 carbon atoms in the chain (a C₂₋₈ alkynyl). In another embodiment, the alkynyl group has 2-4 carbon atoms in the chain (a C₂₋₄ alkynyl). Exemplary alkynyl groups include ethynyl, propynyl, n-butynyl, 2-butynyl, 3-methylbutynyl, n-pentynyl, heptynyl, octynyl and decynyl. An alkylalkynyl is an alkyl group as defined herein bonded to an alkynyl group as defined herein.

An “aryl” group refers to an aromatic monocyclic or multicyclic ring system. In one embodiment, the aryl group has 6-10 carbon atoms. The aryl is optionally substituted with at least one “ring system substituents” and combinations thereof as defined herein. Exemplary aryl groups include phenyl or naphthyl. An alkylaryl is an alkyl group as defined herein bonded to an aryl group as defined herein.

A “heteroaryl” group refers to a heteroaromatic system containing at least one heteroatom ring wherein the atom is selected from nitrogen, sulfur and oxygen. The heteroaryl contains 5 or more ring atoms. The heteroaryl group can be monocyclic, bicyclic, tricyclic and the like. Also included in this definition are the benzoheterocyclic rings. Non-limiting examples of heteroaryls include thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolyl, quinolyl, naphthyridinyl, quinoxalinyl, quinazolinyl, cinnolinyl, pteridinyl, carbolinyl, thiazolyl, oxazolyl, isothiazolyl, isoxazolyl and the like.

An “alkoxy” group refers to an —O-alkyl group wherein R is alkyl as defined above. A “thio” group refers to an —SH group. An “alkylthio” group refers to an —SR group wherein R is alkyl as defined above.

The columnar discotic liquid crystals of the present invention are produced by a self-assembly process. The term “self-assembly” used herein refers to a process of organization of molecules without intervening from an outside source. The self-assembly process takes place in a solution/solvent or directly on the solid-state substrate. In one embodiment, the molecules in a concentration range of 10⁻⁷-10⁻³ M, stack on top of each other in a solution/solvent and form wire-like structures. Depositing a given amount of this solution/solvent, which contain the wire-like structures by, e.g., spin coating, spray coating, Langmuir-Blodgett, zone-casting, or drop casting, results in a wire-like structure that is adsorbed on the surface of the solid-state substrate. The self-assembly manner of formation allows those skilled in the art to obtain nanowires which are uniform in size with less than 10% variability. The parameters for controlling the characteristics of DLC nanowires as well as representative examples of nanowires assembled using different parameters are summarized in Table 1 hereinbelow:

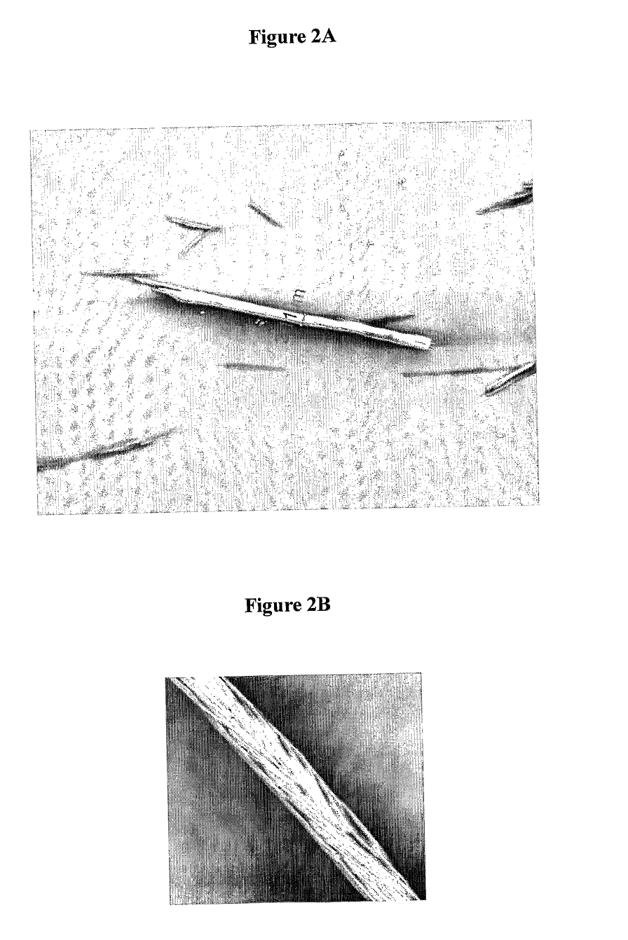

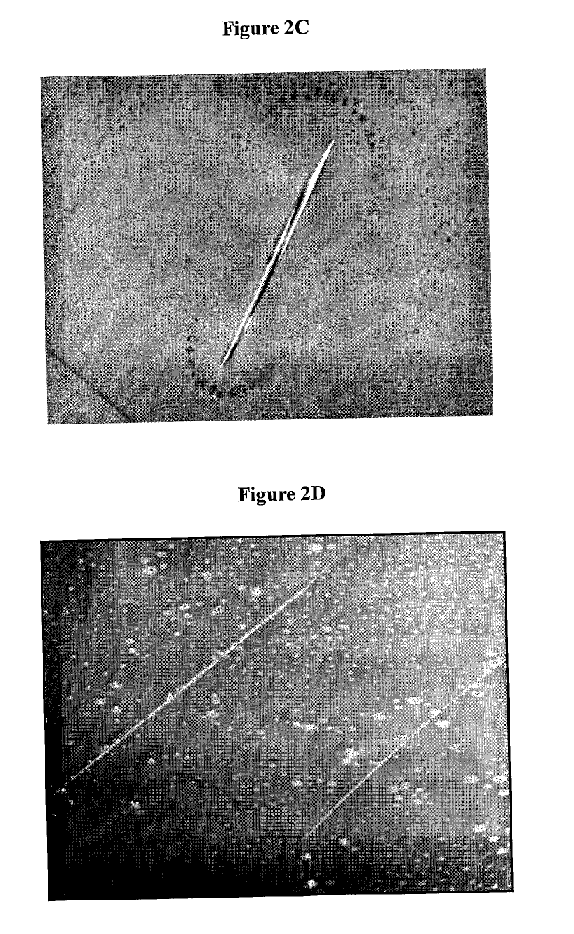

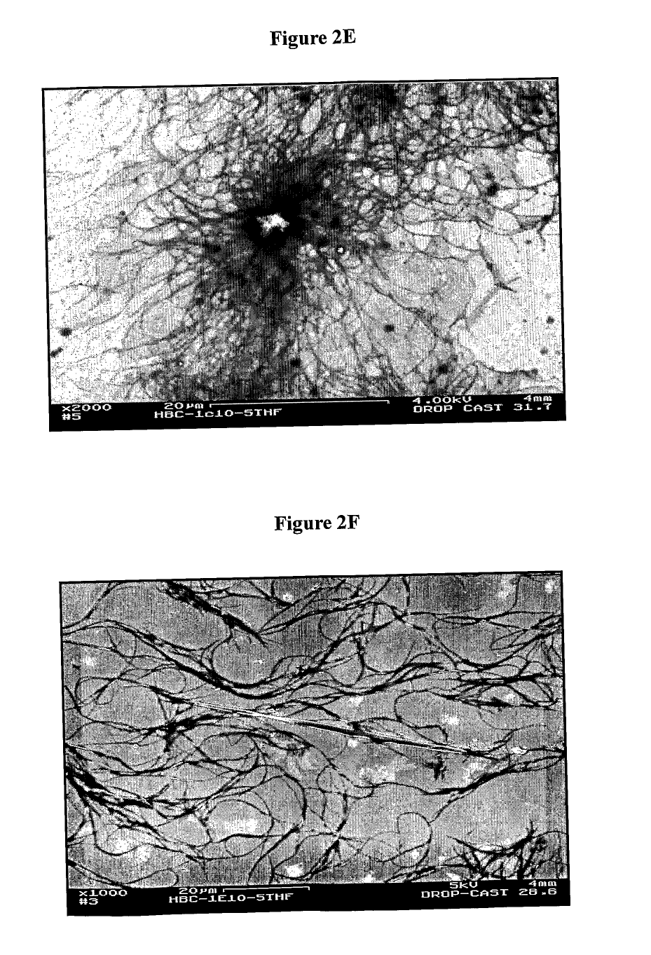

TABLE 1
Summary of the parameters that affect aggregation and morphology of DLC
wires.

| Parameters | | |
|---|---|---|
| Aggregation | Long | FIGS. 2A-2B show an example of an assembly |
| time on surface | (1-48 hrs) | process using: |
| | | Molecule: HBC-C₁₂ |
| | | Solvent: Toluene |
| | | Concentration: 10⁻⁵ M |
| | | Aggregation time: 5-12 hrs (by drop casting) |
| | | FIG. 2A is a SEM micrograph of a nanowire (45 μm × |
| | | 2.11 μm) surrounded by smaller nanowires. |
| | | FIG. 2B is a higher magnification of the nanowire in |
| | | FIG. 2A. |
| | Short | FIG. 2C shows a nanowire (23 μm × 0.66 μm) |
| | (0.5-5 min) | assembled using: |
| | | Molecule: HBC-C₁₂ |
| | | Solvent: Toluene |
| | | Concentration: 10⁻⁵ M |
| | | Aggregation time: 1-2 min (by spin coating) |
| Concentration | Low | FIG. 2D shows an example of an assembly process |
| | (10⁻⁶-10⁻⁷ M) | using: |
| | | Molecule: HBC-C₁₂ |
| | | Solvent: Toluene |
| | | Concentration: 10⁻⁷ M |
| | | Aggregation time: 1-2 min (by spin coating) |
| | | The nanowire in the upper left corner of FIG. 2D has |
| | | dimensions of 82 μm × 0.45 μm. |
| | High | FIG. 2C shows a nanowire (23 μm × 0.66 μm) |
| | (10⁻²-10⁻⁵ M) | assembled using: |
| | | Molecule: HBC-C₁₂ |
| | | Solvent: Toluene |
| | | Concentration: 10⁻⁵ M |
| | | Aggregation time: 1-2 min (by spin coating) |
| Side Groups | Short | FIG. 2E shows an example of an assembly process |
| | (C1-C6) | using: |
| | | Molecule: HBC-C₆ |
| | | Solvent: THF |
| | | Concentration: 10⁻⁵ M |
| | | Aggregation time: 5-12 hrs (by drop casting) |
| | Long | FIG. 2F shows an example of an assembly process |
| | (C7-C48) | using: |
| | | Molecule: HBC-C₁₂ |
| | | Solvent: THF |
| | | Concentration: 10⁻⁵ M |
| | | Aggregation time: 5-12 hrs (by drop casting) |
| | | The nanowire in the center has dimensions of |
| | | 70 μm × 1.5 μm. |
| Solvent | Nonpolar | FIG. 2A shows an example of an assembly process |
| | (0 Debye) | using: |
| | | Molecule: HBC-C₁₂ |
| | | Solvent: Toluene |
| | | Concentration: 10⁻⁵ M |
| | | Aggregation time: 5-12 hrs (by drop casting) |
| | Polar | FIG. 2F shows an example of an assembly process |
| | (>0 Debye) | using: |
| | | Molecule: HBC-C₁₂ |
| | | Solvent: THF |
| | | Concentration: 10⁻⁵ M |
| | | Aggregation time: 5-12 hrs (by drop casting) |

According to another embodiment, the molecules in typical concentration range of 10⁻⁷-10⁻³ M, stay unassembled in the solution/solvent and when deposited on a solid-state substrate, the molecules start to assemble directly on the substrate. In this manner it is possible to obtain both the edge-on (planar) configuration wherein the columns are oriented parallel to the solid substrate, and the face-on (homeotropic) configuration wherein the columns are oriented perpendicular to the solid substrate.

In certain embodiments, the electronic device of the present invention comprises at least one sensor of discotic liquid crystal nanowires for the detection of specific volatile organic compounds. In a second embodiment, the electronic device of the present invention comprises an array of sensors of discotic liquid crystal nanowires. The array of sensors comprises a plurality of sensors between 2 to 1000 sensors, more preferably between 2 to 500 sensors, even more preferably between 2 to 250 sensors, and most preferably between 2 to 125 sensors in an array.

The electronic devices according to the principles of the present invention can be used in any one of the following configurations, namely, as a chemiresistor, a chemicapacitor, or a field effect transistor (FET). In chemiresistors, the electrical resistance of the system is responsive to the presence of a diverse set of analytes in the vapor phase. Sorption of vapor into a chemiresistor induces physical swelling of the material which affects the electron density on the polymeric chains. In chemicapacitors, capacitance of the system is responsive to the presence of a diverse set of analytes in the vapor phase. Sorption of vapor into a chemiresistor induces physical swelling of the material which affects the dielectric constant and charge carrier in the system. In a standard FET, a semiconducting phase is connected to metal source and drain electrodes through which a current is injected and collected, respectively. The conductance of the semiconductor between source and drain is switched on and off by a third gate electrode capacitively coupled through a thin dielectric layer. In the case of p-type (/n-type) semiconductor, applying a positive gate voltage depletes (/accumulates) carriers and reduces (/increases) the conductance, while applying a negative gate voltage leads to an accumulation (/depletion) of carriers and an increase (/decrease) in conductance. The dependence of the conductance on gate voltage makes FETs natural candidates for electrically based sensing devices since the electric field resulting from binding of a charged species to the gate dielectric is analogous to applying a voltage using a gate electrode.

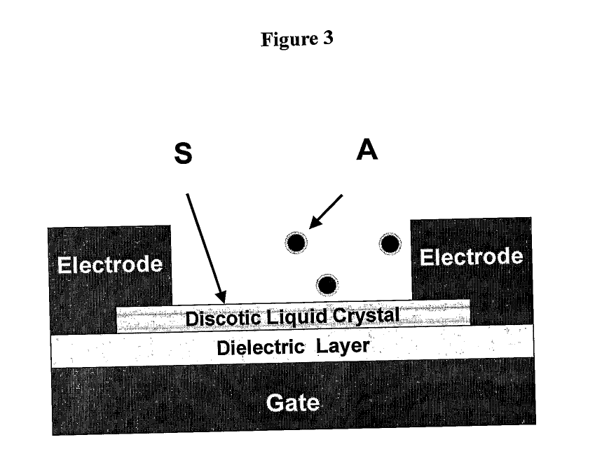

FIG. 3 illustrates the Field Effect Transistor (FET) configuration according to the principles of the present invention. The biomarkers of interest are detected using a system comprising an array of chemically sensitive nanowires of columnar discotic liquid crystals. Two electrodes are attached from each side of the array and the gating is performed from the back. In this configuration, sensing is obtained without a reference electrode. The sensors convert the detection of certain biomarkers into electrical signals, which are conveyed to a pattern recognition analyzer. The pattern recognition analyzer uses algorithms such as neural networks to generate a result.

In currently preferred embodiments, sensing can be performed via spectroscopic ellipsometry. This technique measures the change in polarization upon reflection of polarized light from a surface. Without being bound by any theory or mechanism of action, the adsorption of analyte molecules induces changes in thickness of layers of the DLC nanowires of the present invention. The change in thickness or roughness induces change in polarization which can be recorded by the spectroscopic ellipsometry technique. The signal obtained is subsequently conveyed to a pattern recognition analyzer to generate a result.

According to one embodiment, a method to determine the composition and concentration of VOCs in a sample, comprising exposure of the DLC nanowire sensors of the electronic device to the sample and using pattern recognition algorithms in order to identify and possibly quantify desired VOCs in a given sample, is provided in the present invention. Thus, the present invention further provides a system comprising the electronic device and a pattern recognition analyzer. In practice, the analyzer receives signal outputs or patterns from the device and analyses them by various pattern recognition algorithms to produce an output signature. By comparing an unknown signature with a database of stored or known signatures, VOCs can be identified.

Algorithms for sample analysis, suitable for identifying and possibly quantifying VOCs include, but are not limited to, principal component analysis, Fischer linear analysis, neural network algorithms, genetic algorithms, fuzzy logic pattern recognition, and the like. After analysis is completed, the resulting information can, for example, be displayed on display, transmitted to a host computer, or stored on a storage device for subsequent retrieval.

Many of the algorithms are neural network based algorithms. A neural network has an input layer, processing layers and an output layer. The information in a neural network is distributed throughout the processing layers. The processing layers are made up of nodes that simulate the neurons by the interconnection to their nodes.

In operation, when a neural network is combined with a sensor array, the sensor data is propagated through the networks. In this manner, a series of vector matrix multiplications are performed and unknown analytes can be readily identified and determined. The neural network is trained by correcting the false or undesired outputs from a given input. Similar to statistical analysis revealing underlying patterns in a collection of data, neural networks locate consistent patterns in a collection of data, based on predetermined criteria.

Suitable pattern recognition algorithms include, but are not limited to, principal component analysis (PCA), Fisher linear discriminant analysis (FLDA), soft independent modeling of class analogy (SIMCA), K-nearest neighbors (KNN), neural networks, genetic algorithms, fuzzy logic, and other pattern recognition algorithms. In some embodiments, the Fisher linear discriminant analysis (FLDA) and canonical discriminant analysis (CDA) as well as combinations thereof are used to compare the output signature and the available data from the database.

In other embodiments, principal component analysis is used. Principal component analysis (PCA) involves a mathematical technique that transforms a number of correlated variables into a smaller number of uncorrelated variables. The smaller number of uncorrelated variables is known as principal components. The first principal component or eigenvector accounts for as much of the variability in the data as possible, and each succeeding component accounts for as much of the remaining variability as possible. The main objective of PCA is to reduce the dimensionality of the data set and to identify new underlying variables.

In practice, principal component analysis compares the structure of two or more covariance matrices in a hierarchical fashion. For instance, one matrix might be identical to another except that each element of the matrix is multiplied by a single constant. The matrices are thus proportional to one another. More particularly, the matrices share identical eigenvectors (or principal components), but their eigenvalues differ by a constant. Another relationship between matrices is that they share principal components in common, but their eigenvalues differ. The mathematical technique used in principal component analysis is called eigenanalysis. The eigenvector associated with the largest eigenvalue has the same direction as the first principal component. The eigenvector associated with the second largest eigenvalue determines the direction of the second principal component. The sum of the eigenvalues equals the trace of the square matrix and the maximum number of eigenvectors equals the number of rows of this matrix.

According to some embodiments, the DLC nanowires are self-assembled on top of the dielectric layer. According to other embodiments, the DLC nanowires are self-assembled in solution following by their adsorption to the dielectric layer to form e.g. Field Effect Transistor (FET).

In certain embodiments, the DLC nanowires are used in an edge-on configuration (planar orientation). The term “edge-on” or “planar orientation” as used herein refers to a configuration wherein the columns of the DLCs are oriented parallel to the solid substrate. As used herein, this term further denotes a configuration wherein the nanowire is laid on top of the solid substrate with its long axis parallel to the substrate. In this manner, the target molecules adsorb on the surface of the DLC nanowires. Changes in the resistance, capacitance, and field effect of these nanowires upon target adsorption are recorded and translated into a sensing signal. The edge-on configuration was hitherto known to be more suitable for fabricating photovoltaic devices (see e.g. PCT Application No. WO 04/075313).

According to the principles of the present invention, the edge-on configuration provides the fabrication of either one of a chemiresistor, a chemicapacitor, or a field effect transistor (FET) sensing devices with advantageous sensing capabilities. The edge-on configuration of the present invention having electrodes at either end of the DLC nanowires provide sensitivity of less than one part per million (ppm) for volatile organic compounds to be detected.

Columnar discotic liquid crystal nanowires are more readily appropriate for miniaturization and for fabricating highly sensitive sensors. The decrease of size increases both the surface area as well as the electric performance of these discotic liquid crystals, as compared to 2D films. In contrast to other sensors, the DLC nanowires can simultaneously be highly sensitive, relatively simple for preparation, stable, low power, lightweight, fast-response, and allow simple signal transduction.

According to one embodiment, a method to determine the composition and concentration of VOCs in a sample, comprising exposure of the DLC nanowire sensors of the electronic device to the sample and using pattern recognition algorithms in order to identify and possibly quantify the components composing a sample is provided in the invention. This method is useful especially, but not exclusively, in the fields of medicine, food quality control, environmental monitoring, and explosives.

This method is especially valuable to diagnose a disease in a subject. The method is applicable to the headspace of bodily secretions such as, but not limited to, serum, urine, feces, vaginal discharge, sperm, saliva etc. The system is able to detect VOCs in breath directly exhaled by the subject on the device, without a need for pre-concentrating or dehumidifying the sample. Other possibilities include exhaling into an inert bag and then exposing the collected breath to the electronic nose device.

In a particular embodiment, the method described herein is used to diagnose cancer. Gas-Chromatography linked with Mass-Spectrometry (GC-MS) studies have shown that volatile C₄-C₂₀ alkanes and certain monomethylated alkanes as well as benzene derivatives appear to be elevated in various instances of cancer. The compounds of interest are generally found in the range of 1-20 ppb in healthy human breath, but can be seen in distinctive mixture compositions at elevated levels in the range of 10-100 ppb in the breath of diseased patients. The VOC levels are elevated even at the early stages of the disease, since they reflect a change in human body chemistry. This change appears regardless of the cancerous tumor size. In addition, biomarkers of a specific disease (e.g., lung cancer) possess distinctive mixture compositions/patterns in comparison to biomarkers of other diseases even those of closely related diseases (e.g., breast cancer). Thus, using the methods of the present invention would allow the discrimination between different types of diseases.

In one embodiment, the present invention relates to the diagnosis of cancer using the electronic nose device/system disclosed herein. The term “cancer” refers to a disorder in which a population of cells has become, in varying degrees, unresponsive to the control mechanisms that normally govern proliferation and differentiation. Cancer refers to various types of malignant neoplasms and tumors, including metastasis to different sites. Non-limiting examples of cancers which can be detected by the electronic device/system of the present invention are brain, ovarian, colon, prostate, kidney, bladder, breast, lung, oral, and skin cancers. Specific examples of cancers are: adenocarcinoma, adrenal gland tumor, ameloblastoma, anaplastic tumor, anaplastic carcinoma of the thyroid cell, angiofibroma, angioma, angiosarcoma, apudoma, argentaffinoma, arrhenoblastoma, ascites tumor cell, ascitic tumor, astroblastoma, astrocytoma, ataxia-telangiectasia, atrial myxoma, basal cell carcinoma, benign tumor, bone cancer, bone tumor, brainstem glioma, brain tumor, breast cancer, vaginal tumor, Burkitt's lymphoma, carcinoma, cerebellar astrocytoma, cervical cancer, cherry angioma, cholangiocarcinoma, a cholangioma, chondroblastoma, chondroma, chondrosarcoma, chorioblastoma, choriocarcinoma, larynx cancer, colon cancer, common acute lymphoblastic leukaemia, craniopharyngioma, cystocarcinoma, cystofibroma, cystoma, cytoma, ductal carcinoma in situ, ductal papilloma, dysgerminoma, encephaloma, endometrial carcinoma, endothelioma, ependymoma, epithelioma, erythroleukaemia, Ewing's sarcoma, extra nodal lymphoma, feline sarcoma, fibroadenoma, fibrosarcoma, follicular cancer of the thyroid, ganglioglioma, gastrinoma, glioblastoma multiforme, glioma, gonadoblastoma, haemangioblastoma, haemangioendothelioblastoma, haemangioendothelioma, haemangiopericytoma, haematolymphangioma, haemocytoblastoma, haemocytoma, hairy cell leukaemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angiofibroma, Kaposi sarcoma, kidney tumour, large cell lymphoma, leukemia, chronic leukemia, acute leukemia, lipoma, liver cancer, liver metastases, Lucke carcinoma, lymphadenoma, lymphangioma, lymphocytic leukaemia, lymphocytic lymphoma, lymphocytoma, lymphoedema, lymphoma, lung cancer, malignant mesothelioma, malignant teratoma, mastocytoma, medulloblastoma, melanoma, meningioma, mesothelioma, metastatic cancer, Morton's neuroma, multiple myeloma, myeloblastoma, myeloid leukemia, myelolipoma, myeloma, myoblastoma, myxoma, nasopharyngeal carcinoma, nephroblastoma, neuroblastoma, neurofibroma, neurofibromatosis, neuroglioma, neuroma, non-Hodgkin's lymphoma, oligodendroglioma, optic glioma, osteochondroma, osteogenic sarcoma, osteosarcoma, ovarian cancer, Paget's disease of the nipple, pancoast tumor, pancreatic cancer, phaeochromocytoma, pheochromocytoma, plasmacytoma, primary brain tumor, progonoma, prolactinoma, renal cell carcinoma, retinoblastoma, rhabdomyosarcoma, rhabdosarcoma, solid tumor, sarcoma, secondary tumor, seminoma, skin cancer, small cell carcinoma, squamous cell carcinoma, strawberry haemangioma, T-cell lymphoma, teratoma, testicular cancer, thymoma, trophoblastic tumor, tumourigenic, vestibular schwannoma, Wilm's tumor, or a combination thereof.

The system of the present invention can further help diagnose other medical disorders including, but not limited to, acute asthma, hepatic coma, rheumatoid arthritis, schizophrenia, ketosis, cardiopulmonary disease, uremia, diabetes mellitus, dysgeusia/dysosmia, cystinuria, cirrhosis, histidinemia, tyrosinemia, halitosis, and phenylketonuria.

Due to the miniaturized dimensions of the electronic nose devices (in the range of 10-100 nanometers to a few micrometers), these devices could be installed in any electronic apparatus. For example, these devices could be integrated in a watch or cellular phone, as a warning system for the start of an infection or other disease in the body of an individual.

According to other embodiments, the device/system of the present invention could be used for the detection of spoilage in food products via the determination of the composition and concentration of VOC in a food sample. Information regarding early infectious and toxic agents can be gleaned using the device of the present invention, in food production chains. According to another embodiment, the proposed technology could enable efficient warning of pollutions in water and air. These embodiments allow the use of the present invention for environmental monitoring.

The principles of the present invention are demonstrated by means of the following non-limitative examples.

### EXAMPLES

### Example 1

### Composition and Synthesis of the Columnar Discotic Liquid Crystals

The columnar discotic liquid crystals of the present invention were produced from molecules provided from the group of Prof. Klaus Mullen, Max-Planck Institute for Polymer Research, Mainz, Germany. In particular, hexa-peri-hexabenzocoronene (HBC) molecules having the following functional groups: HBC-C₆₋₂, HBC-C_{10,6}, HBC-C₁₄₋₁₀, and HBC-C₁₂ were synthesized according to methods well known in the art, see e.g. Watson et al., J. Am. Chem. Soc., 126(5), 1402, 2004; Pisula et al., J. Am. Chem. Soc., 126(26), 8074, 2004; Wang & Mullen, J. Org. Chem., 69(24), 8194, 2004.

### Example 2

### Self-Assembly of the Discotic Liquid Crystal Nanowires

The length and diameter of the DLC nanowires of the present invention is dependant on various experimental parameters, such as the concentration level of the molecules, the type of the solvent/solution, and the deposition mode. In particular, the molecules in a concentration range of 10⁻⁷-10⁻³ M self-assemble in any one of the following manners:
1. molecules are stacked one on top of the other in solution/solvent to form wire-like structures, followed by deposition on a substrate using methods such as spin coating, spray coating, Langmuir-Blodgett, zone-casting, or drop casting, well known in the art.
2. molecules stay unassembled in solution/solvent and are consequently assembled namely stacked one on top of the other directly on the substrate.

Controlling the dimensions of the nanowire is performed by varying the manner in which self-assembly is performed or alternatively by tailoring parameters such as the concentrations of molecules, and the deposition mode. For example, drop casting of a 10⁻⁵ M HBC-C₁₂ solution in toluene on a solid-state substrate, produce wires that are 700 nm in diameter and 10 μm in length. Alternatively, spin coating of a 10⁻⁵ M HBC-C₁₂ solution in toluene on a solid-state substrate, produce wires that are 60 nm in diameter and 6 μm in length. Similarly, spin coating of 10⁻⁶ M HBC-C₁₂ solution in toluene on a solid-state substrate, produce wires that are 20 nm in diameter and 1 μm in length.

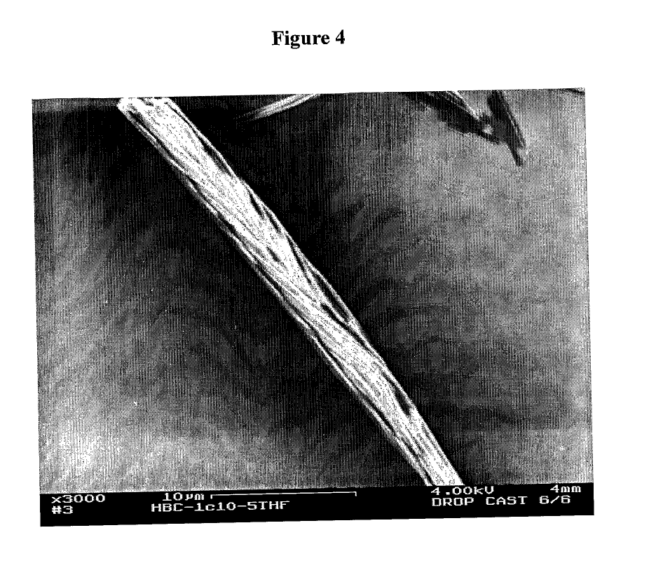

To produce an ensemble of cylindrical nanowires having equivalent dimensions to the above mentioned, a self-assembly procedure was used wherein the individual nanowires were congregated edge-to-edge to form wires having overall larger diameters (FIG. 4).

### Example 3

### Fabrication of the Electronic Device

Discotic liquid crystal nanowires produced as described hereinabove, are dispersed from solution/solvent (e.g., THF or toluene) onto a doped silicon substrate containing a thin film of dielectric layer mainly composed of SiO₂ and ZrO₂.

The contacts to the DLC nanowires are performed by electron beam lithography followed by evaporation of a metal that forms an ohmic contact (Stern, J. Vac. Sci. Technol. B, 24, 231, 2006). The electrodes for measuring the electrical properties of the deposited discotic liquid crystals are produced by various processes well known in the art which include, but are not limited to, photolithography, e-beam lithography, focused ion beam (FIB), direct evaporation/sputtering through shadow mask, and soft stamp contact.

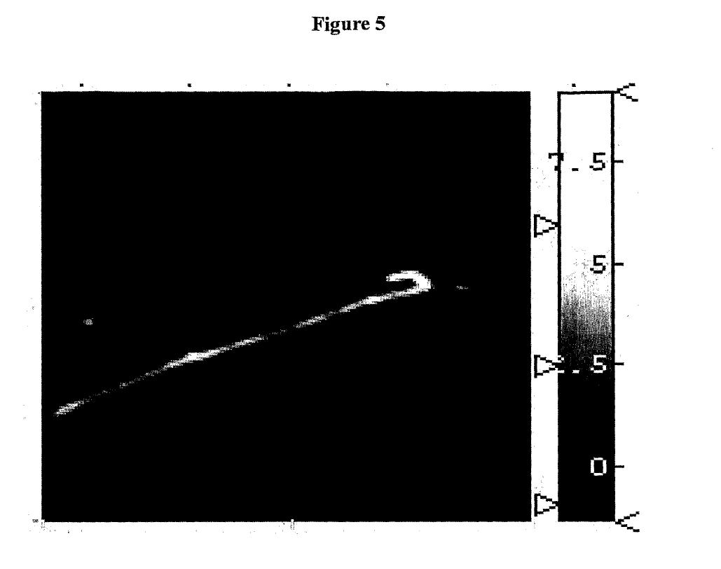

The electronic devices of the present invention are fabricated in an edge-on configuration, wherein the DLC nanowires are laid down on the substrate allowing the electrodes to be connected at either end of the DLC nanowires. FIG. 5 shows an atomic force micrograph of an exemplary DLC nanowire which is laid down in an edge-on configuration.

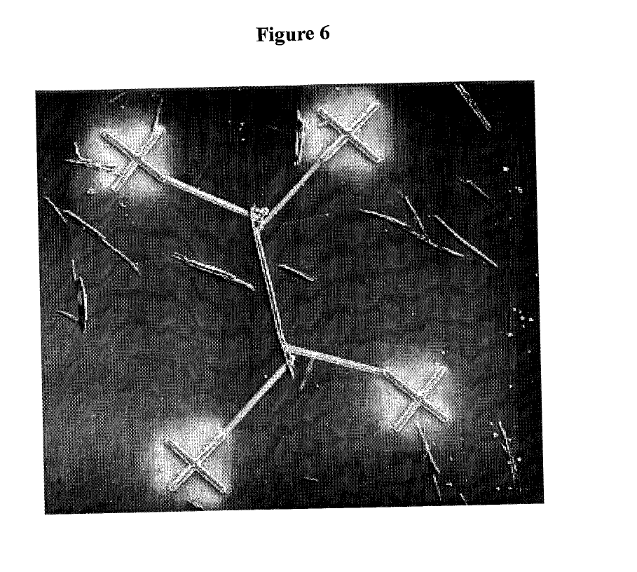

A DLC nanowire that is contacted by means of Focused Ion Beam (FIB) to form Field Effect Transistor (FET) is shown in FIG. 6. The fabrication process was performed by initial selection of a DLC nanowire, which was deposited on the substrate, using the FIB's Scanning Electron Microscope (SEM). This was followed by the deposition of a metal, preferably platinum (Pt) using the SEM electron beam having a current of 1.6 nano-amps and voltage of 5 kilo-volts, for each of the electrodes contacting the DLC nanowire. The number of electrodes was ranged from two electrodes to four electrodes. The typical dimensions of the contacts obtained were 20 μm in length, 1 μm in width and approximately 500 nm in thickness. Each contact was connected to a pad far from the DLC nanowire that was designated for contacting the DLC nanowire, to obtain the electrical signal desired for device operation. The pads were fabricated by bombardment of Gallium ions with a current of 0.46 nano-amps and a voltage of 30 kilo-volts. Typical dimension that were obtained for the pad are 10×20 μM.

### Example 4

### Electrical Stability in Humid Environments

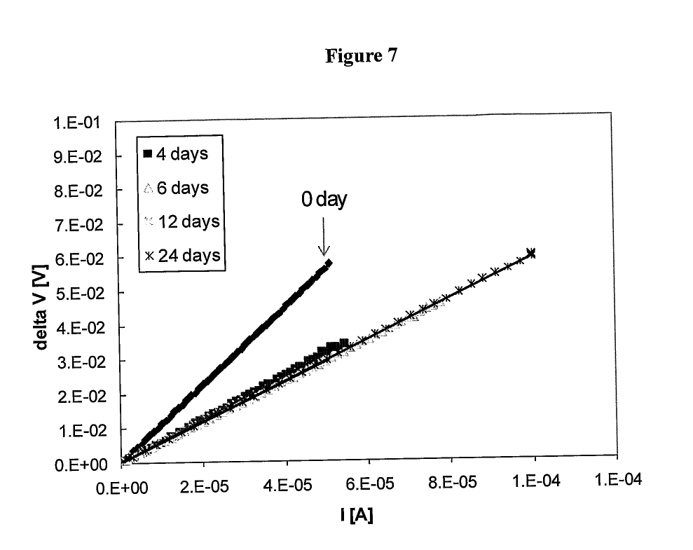

In order to verify the electronic stability of such devices under humid environments, electrical measurements of a device similar to that shown in FIG. 6 were performed. FIG. 7 shows voltage (V) vs. current (I) correlations of the device, measured at different time intervals. Each time interval corresponded to a different percentage of humidity, in the range of 40-80% relative humidity (RH). The device showed high stability in humid environment after a short exposure time. In particular, for all samples, voltage vs. current (V vs. I) data were linear and symmetric about zero bias. The voltage vs. current characteristics obtained by either two- or four-point probe methods showed no hysteresis, indicating the absence of charge accumulation and/or “electronic memory” effects. Moreover, the conductivity of the wires remained stable, after one day of exposure with approximately 95% performance in comparison to initial values. Following the minor reduction in performance after one day of exposure, the device showed no dependency on exposure time in ambient conditions; neither did it show a dependency on the percentage of humidity.

### Example 5

### Spectroscopic Ellipsometry (SE) Characterization During Exposure to the Analytes

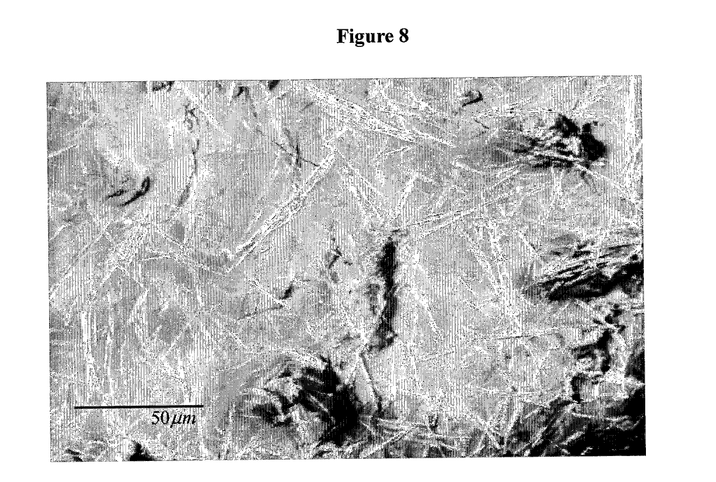

The DLC nanowires of the present invention were prepared by drop casting HBC-C₁₂ on a Si\SiO₂ substrate (FIG. 8). In particular, pieces (1×3 cm²) of degenerative p-doped silicon (100) wafer having an oxide layer (SiO₂) of approximately 100 nm were used as substrates. The DLC nanowires were prepared by drop casting a solution of HBC-C₁₂ (10 μl of 10⁻⁴M in toluene). The samples were dried for approximately 1 hour in air at ambient temperature, followed by drying on a hot plate at 70° C. Additional Si\ SiO₂ substrate was taken from the same wafer as a reference.

A SEM micrograph shows that the HBC nanowires possess large, sponge-like agglomerates (FIG. 8). These structures have diameters ranging from 1 to 2 μms, and typically several tens of microns in length. The HBC edge-on nanowires cover about ⅕ of the surface.

The changes in thickness and refractive index during exposure to analytes were monitored by spectroscopic ellipsometry (SE). Spectra were recorded over a range of 250-1700 nm at an incidence angle of 70°, using a spectroscopic phase modulated ellipsometer (M-2000U Automated Angle, J. A. Woollam Co., Inc., USA). A CCD camera was used for fast monitoring. The measurements were performed at time intervals of 2-10 seconds, depending on the desired accuracy. Samples were mounted in an air-tight triangular exposure cell, designed by J. A. Woollam Co. Inc., to assure that the presence of windows do not interfere with the measurement (i.e. do not change light polarization). The exposure cell was connected to a home-build flow system flushed with dry air introducing bubblers of liquid analytes and a bypass for dry air free of analytes. The concentration of analytes in the dry air was regulated via their vapor pressure. Water and methanol were used as polar analytes while octane and decane were used as apolar analytes.

The exact thickness of the SiO₂ layer was determined experimentally for every substrate prior to the deposition of the HBC structures and the optical constants of Si/SiO₂ were taken from Arwin & Aspnes, Thin Sol. Films, 113, 101, 1984.

In order to evaluate “thickness changes” upon analyte adsorption a homogenous, isotropic HBC layer of 500 nm thickness was assumed. The optical constants were then determined point-by-point from the experimental spectra wherein a macroscopic area of about 1×7 mm² is measured at each time using an incidence angle of 70°. The highly optically anisotropic HBC structures were represented in macroscopic average as an isotropic material having normal Gaussian distribution. Importantly the samples were not moved during a full cycle of SE measurements to avoid artifacts due to local variations of the HBC layer.

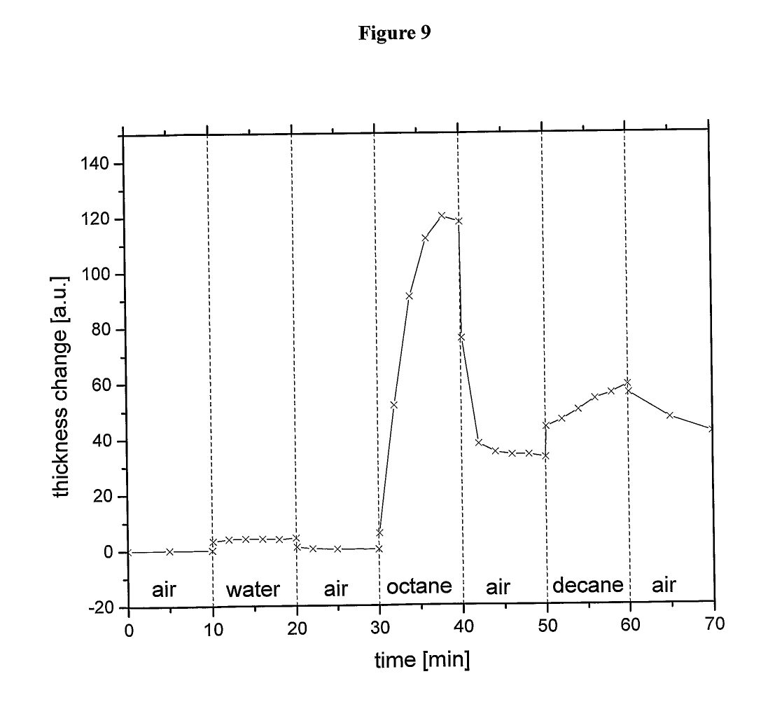

FIG. 9 summarizes the extracted changes in “thickness” (in arbitrary units) during the exposure to water, octane and decane. These changes correspond to physical modification of the HBC nanowires due to adsorption of the analytes, either on the surface or in between the constituent HBC nanowires. FIG. 9 shows that the exposure to water caused a very small and practically negligible increase in thickness. This change was further shown to be fully and rapidly reversible, even after 40 minutes of exposure to water. In contrast, the exposure to octane caused a dramatic increase in thickness, which was not fully reversible after subsequent flushing with dry air for 10 minutes. This increase in thickness was shown to be reproducible. The exposure to decane caused a measurable and reproducible change in the thickness as well. This change was only slowly reversible. Interestingly, the exposure to methanol caused a measurable decrease in thickness. Without being bound by any theory or mechanism of action, methanol molecules may be adsorbed in between HBC nanowires to interact with the C₁₂ side groups to introduce aggregation and a decrease in the distance between adjacent chains.

In order to confirm that the observed changes are not due to substrate effects or the adsorption of analytes on windows of the exposure cell, Si/SiO₂ reference sample with no HBC nanowires was exposed to the same analytes and SE measurements were performed. Using the reference sample, only very small variations of the SE spectra were observed in comparison to the pronounced changes in the spectra obtained for samples having the HBC nanowires. SE measurements over an interval of 24 hours while flushing the HBC samples in the exposure cell with dry air further confirmed that the observed changes were not due to the redistribution or reduction of the HBC nanowires under flow.

These experiments clearly show that the exposure to analytes and in particular to apolar analytes causes significant changes in thickness and optical properties of the HBC nanowire layers. In contrast, the effect of water exposure is negligible. SE thus allow “real time” monitoring of the physical changes of the HBC structures during exposure to analytes, including an assessment of the reversibility of this process. It is further shown that changes in thickness are characteristic of the analytes wherein apolar analytes (decane or octane) induce a different optical behavior than polar analytes (methanol).

It is appreciated by persons skilled in the art that the present invention is not limited by what has been particularly shown and described hereinabove. Rather the scope of the present invention includes both combinations and sub-combinations of various features described hereinabove as well as variations and modifications. Therefore, the invention is not to be constructed as restricted to the particularly described embodiments, and the scope and concept of the invention will be more readily understood by references to the claims, which follow.

1. An electronic device comprising at least one chemically sensitive sensor for the detection of volatile organic compounds; wherein the chemically sensitive sensor comprises at least one nanowire of columnar discotic liquid crystals; and wherein the electronic device is essentially not sensitive to humidity.

2. The electronic device according to claim 1, comprising at least one chemically sensitive sensor for the detection of volatile organic compounds; wherein the chemically sensitive sensor consists essentially of at least one nanowire of columnar discotic liquid crystals; and wherein the electronic device is essentially not sensitive to humidity.

3. (canceled)

4. (canceled)

5. The electronic device according to claim 1, wherein the at least one nanowire of columnar discotic liquid crystals is assembled from hexa-peri-hexabenzocoronene (HBC) molecules selected from the group consisting of: HBC-C_{6,2}, HBC-C_{10,6}, HBC-C_{14,10}, and HBC-C₁₂.

6. The electronic device according to claim 1, wherein the at least one nanowire of columnar discotic liquid crystals is assembled from molecules selected from the group consisting of: hexa-n-alkoxytriphenylenes [C₁₈H₆(—O—CₙH₂ₙ₊₁)₆], hexa-n-alkylthiotriphenylenes [C₁₈H₆(—S—CₙH₂ₙ₊₁)₆], and hexa-n-alkoxyaryltriphenylenes [C₁₈H₆(C₆H₃(—O—CₙH₂ₙ₊₁)₆], wherein n=5, 6, 8, 11, or 13.

7. The electronic device according to claim 1, wherein the at least one nanowire of columnar discotic liquid crystals is assembled from hexa-alkyl-substituted derivatives of hexabenzo[bc,ef,hi,kl,no,qr]coronene [C₄₂H₁₂—(CₙH₂ₙ₊₁)₆], wherein n=5, 6, 8, or 11.

8. The electronic device according to claim 1, wherein the at least one nanowire of columnar discotic liquid crystals is assembled from hexa-alkylaryl-substituted derivatives of hexabenzo-[bc,ef,hi,kl,no,qr]coronene [C₄₂H₁₂(C₆H₄(—CₙH₂ₙ₊₁))₆], wherein n=5, 6, or 12.

9. The electronic device according to claim 1, wherein the at least one nanowire of columnar discotic liquid crystals is assembled from molecules selected from the group consisting of 2,3,6,7,10,11-hexakis[3,4-bis(alkoxy)-phenyl]triphenylenes [C₁₈H₆(C₆H₃(—O—CₙH₂ₙ₊₁)₂)₆], 6,6′,6″,7,7′,7″,10,10′,10″,11,11′,11″-dodecaalkoxy-2,3′:3,2″:2′,3″tris(triphenylenylenes) [C₅₄H₁₈(—OCₙH₂ₙ₊₁)₁₂], wherein n=6, 8, 10, and 12.

10. The electronic device according to claim 1, fabricated in a configuration selected from the group consisting of a chemiresistor, a chemicapacitor, and a field effect transistor.

11. The electronic device according to claim 1, wherein the at least one nanowire of columnar discotic liquid crystals is in planar orientation.

12. A system having:
an electronic device comprising an array of chemically sensitive sensors for the detection of volatile organic compounds; wherein the chemically sensitive sensors comprise nanowires of columnar discotic liquid crystals, and wherein the electronic device is essentially not sensitive to humidity, and
a pattern recognition analyzer, wherein the pattern recognition analyzer receives sensor output signals and compares them to stored data.

13. (canceled)

14. (canceled)

15. The system according to claim 12, wherein the nanowires of columnar discotic liquid crystals are assembled from hexa-peri-hexabenzocoronene (HBC) molecules selected from the group consisting of: HBC-C_{6,2}, HBC-C_{10,6}, HBC-C_{14,10}, and HBC-C₁₂.

16. The system according to claim 12, wherein the nanowires of columnar discotic liquid crystals are assembled from molecules selected from the group consisting of: hexa-n-alkoxytriphenylenes [C₁₈H₆(—O—CₙH₂ₙ₊₁)₆], hexa-n-alkylthiotriphenylenes [C₁₈H₆(—S—CₙH₂ₙ₊₁)₆], and hexa-n-alkoxyaryltriphenylenes [C₁₈H₆(C₆H₃(—O—CₙH₂ₙ₊₁)₂]₆], wherein n=5, 6, 8, 11, or 13.

17. The system according to claim 12, wherein the nanowires of columnar discotic liquid crystals are assembled from hexa-alkyl-substituted derivatives of hexabenzo[bc,ef,hi,kl,no,qr]coronene [C₄₂H₁₂—(CₙH₂ₙ₊₁)₆], wherein n=5, 6, 8, or 11.

18. The system according to claim 12, wherein the nanowires of columnar discotic liquid crystals are assembled from hexa-alkylaryl-substituted derivatives of hexabenzo-[bc,ef,hi,kl,no,qr]coronene [C₄₂H₁₂(C₆H₄(—CₙH₂ₙ₊₁))₆], wherein n=5, 6, or 12.

19. The system according to claim 12, wherein the nanowires of columnar discotic liquid crystals are assembled from molecules selected from the group consisting of: 2,3,6,7,10,11-hexakis[3,4-bis(alkoxy)-phenyl]triphenylenes [C₁₈H₆(C₆H₃(—O—CₙH₂ₙ₊₁)₂)₆], 6,6′,6″,7,7′,7″,10,10′,10″, 11,11′,11″-dodecaalkoxy-2,3′:3,2″:2′,3″tris(triphenylenylenes) [C₅₄H₁₈(—OCₙH₂ₙ₊₁)₁₂], wherein n=6, 8, 10, and 12.

20. The system according to claim 12, wherein the electronic device is fabricated in a configuration selected from the group consisting of a chemiresistor, a chemicapacitor, and a field effect transistor.

21. The system according to claim 12, wherein the nanowires of columnar discotic liquid crystals are in planar orientation.

22. The system according to claim 12, wherein the pattern recognition analyzer comprises at least one algorithm selected from the group consisting of artificial neural network algorithms, principal component analysis (PCA), multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, and nearest neighbor.

23. (canceled)

24. A method for diagnosing a disease in a subject, comprising the steps of
a) collecting a sample comprising volatile organic compounds selected from exhaled breath and the headspace of a container in which at least one bodily fluid or secretion of the subject have been placed;
b) providing a system comprising an electronic device for detecting volatile organic compounds according to claim 12;
c) exposing the sensor array of the electronic device to the sample; and
d) using pattern recognition algorithms to determine the composition and concentration of selected volatile organic compounds indicative of a disease in the sample.

25. The method according to claim 24, wherein the disease is selected from the group consisting of cancer, acute asthma, hepatic encephalopathy, rheumatoid arthritis, schizophrenia, ketosis, cardiopulmonary disease, uremia, diabetes mellitus, larynx cancer, dysgeusia/dysosmia, cystinuria, cirrhosis, histidinemia, tyrosinemia, halitosis and phenylketonuria.

26. (canceled)

27. The method according to claim 24, wherein the bodily fluid or secretion is selected from the group consisting of serum, urine, feces, sweat, vaginal discharge, saliva and sperm.

28. The method according to claim 24, wherein the pattern recognition analyzer comprises at least one algorithm selected from the group consisting of artificial neural network algorithms, principal component analysis (PCA), multilayer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, and nearest neighbor.

29. A method for determining at least one of the composition and concentration of selected volatile organic compounds in a sample, comprising the steps of:
a) providing a system comprising an electronic device for detecting volatile organic compounds according to claim 12;
b) exposing the sensor array of the electronic device to the sample; and
c) using pattern recognition algorithms to detect the presence of the volatile organic compounds and measure their concentration in the sample.

30. The method according to claim 29, wherein the detection of the volatile organic compounds comprises the use of spectroscopic ellipsometry.

31. The method according to claim 29 for detecting spoilage in food products or for detecting environmental pollution in water or air.

32-45. (canceled)
